(19) United States
(12) Patent Application Publication
(10) Pub. No.: US 2023/0055386 A1
(43) Pub. Date: Feb. 23, 2023

(54) ROBOTIC SURGERY SYSTEM

(71) Applicant: Titan Medical Inc., Toronto (CA)

(72) Inventors: Aki Hannu Einari Laakso, Raleigh, NC (US); Hans Christian Pflaumer, Apex, NC (US); James Davies, Cambridge (GB); Adrian Edward Cooper, Cambridgeshire (GB); Thomas Henry Brittain, Bury St Edmunds (GB); Andre Fraser Monteiro, Cambridgeshire (GB); Chace Francis Medeiros, Raleigh, NC (US)

(21) Appl. No.: 17/820,392

(22) Filed: Aug. 17, 2022

Related U.S. Application Data
(60) Provisional application No. 63/260,481, filed on Aug. 20, 2021.

(51) Int. Cl. A61B 34/30; A61B 50/13

(52) U.S. Cl. CPC A61B 34/30; A61B 50/13; A61B 2034/306; A61B 2034/301; A61B 2090/066

(57) ABSTRACT

A robotic surgical system is provided with a central drive unit that supports and operates one or more robotic tools and a robotic arm and boom assembly that movably supports the control unit assembly in space. The robotic arm and boom assembly selectively allows movement of the control unit assembly along a plane, as well as in pitch and yaw, upon actuation of one or more actuators of the robotic arm and boom assembly to allow movement of the central drive unit.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The present disclosure generally relates to robotic surgical systems, and more particularly to mechanisms for moving an arm assembly and control unit assembly of a robotic surgical system.

### Description of the Related Art

Robotic surgery systems generally include an operator interface that receives operator input from a surgeon and causes corresponding movements of surgical tools within a body cavity of a patient to perform a surgical procedure. The operator interface can be on a workstation that the surgeon interfaces with to perform a surgical procedure using the surgical tools. The surgical tools can be on a cart separate from the workstation. The cart can be mobile, allowing hospital staff to move the cart into an operating room prior to the surgical procedure, and to remove it from the operating room once the surgical procedure has been completed.

### SUMMARY

In accordance with one aspect of the disclosure, a robotic surgical system is provided with a central drive unit that supports and operates one or more robotic tools and a robotic arm and boom assembly that movably supports the control unit assembly in space. The robotic arm and boom assembly selectively allows movement of the control unit assembly along a plane, as well as in pitch and yaw, upon actuation of one or more actuators of the robotic arm and boom assembly to allow movement of the central drive unit.

In accordance with another aspect of the disclosure, a robotic surgical system has an arm assembly constrained to move in a horizontal direction and one or more joints for adjusting one or more of a lateral position, a pitch and a yaw of a central drive unit to thereby adjust a respective pitch and yaw of the surgical instruments and endoscope removably coupled to the central drive unit based on operator input.

In accordance with another aspect of the disclosure, a robotic surgical system is operable to adjust a location of a remote center of motion along an axis of an insertion tube to maintain the remote center of motion at an incision location of a patient while allowing a length of the insertion tube that extends inside the patient to be adjusted to thereby adjust an insertion depth of the surgical instruments and endoscope in a surgical space within the patient based on operator input.

In accordance with another aspect of the disclosure, a robotic surgical system is provided. The system includes a cart extending vertically above a base. The system also includes an arm assembly movably coupled to the cart, the arm assembly selectively movable relative to the cart to vary a height of the arm assembly relative to the base. The arm assembly is constrained to move horizontally and includes a plurality of transverse arms extending perpendicular to the cart. Each transverse arm is pivotally coupled to another of the transverse arms via a joint and configured to pivot about a vertical axis through the joint. A vertical arm is pivotally coupled to a last of the transverse arms about a yaw joint and extending downwardly therefrom. A central drive unit is pivotally coupled to the vertical arm about a pitch joint, the central drive unit comprising one or more robotic surgical instruments and an endoscope removably coupled thereto. The surgical instruments and endoscope are configured to extend through a single insertion tube configured to be inserted through an incision location in a patient. Each of the joint, yaw joint and pitch joint is robotically controlled to adjust one or more of a lateral position, a pitch and a yaw of the central drive unit to thereby adjust a respective pitch and yaw of the surgical instruments and endoscope based on operator input, and to adjust a location of a remote center of motion along an axis of the insertion tube to maintain the remote center of motion at the incision location while allowing a length of the insertion tube that extends inside the patient to be adjusted to thereby adjust an insertion depth of the surgical instruments and endoscope in a surgical space within the patient based on operator input.

In accordance with another aspect of the disclosure, a robotic surgical system is provided. The system includes a cart extending vertically above a base. The system also includes an arm assembly movably coupled to the cart, the arm assembly selectively movable relative to the cart to vary a height of the arm assembly relative to the base. The arm assembly is constrained to move horizontally. The arm assembly includes a first transverse arm vertically movable relative to the cart and at least partially extending along a first plane. A second transverse arm is pivotally coupled to the first transverse arm about a first joint and extending along a second plane parallel to the first plane, the second transverse arm configured to pivot about a vertical axis through the first joint. A third transverse arm is pivotally coupled to the second transverse arm about a second joint and extending along a third plane parallel to the first and second planes, the third transverse arm configured to pivot about a vertical axis through the second joint. A vertical arm is pivotally coupled to the third transverse arm about a fourth joint and extending downwardly therefrom. A central drive unit is pivotally coupled to the vertical arm about a fifth joint. The central drive unit comprises one or more robotic surgical instruments and an endoscope removably coupled thereto. The surgical instruments and endoscope are configured to extend through a single insertion tube configured to be inserted through an incision location in a patient. Each of the joints is robotically controlled to adjust one or more of a lateral position, a pitch and a yaw of the central drive unit to thereby adjust a respective pitch and yaw of the surgical instruments and endoscope based on operator input, and to adjust a location of a remote center of motion along an axis of the insertion tube to maintain the remote center of motion at the incision location while allowing a length of the insertion tube that extends inside the patient to be adjusted to thereby adjust an insertion depth of the surgical instruments and endoscope in a surgical space within the patient based on operator input.

In accordance with another aspect of the disclosure, a robotic surgical system is provided. The system includes a cart extending vertically above a base. An arm assembly is movably coupled to the cart, the arm assembly selectively movable relative to the cart via a boom arm that connects the arm assembly to the cart to vary a height of the arm assembly relative to the base. The arm assembly is pivotally coupled to the boom arm via a first joint and configured to pivot about a vertical axis through the first joint. The arm assembly includes a plurality of transverse arm sections extending perpendicular to the cart. Each transverse arm section is telescopically coupled to another of the transverse arm sections and operable by one or more actuators to linearly extend relative thereto between an extended position and a retracted position. A vertical arm pivotally coupled to a last of the transverse arm sections about a second joint and extending downwardly therefrom. A central drive unit is pivotally coupled to the vertical arm portion about a third joint. The central drive unit comprises one or more robotic surgical instruments and an endoscope removably coupled thereto, the surgical instruments and endoscope configured to extend through a single insertion tube configured to be inserted through an incision location in a patient. Each of the joints and actuators is robotically controlled to adjust one or more of a lateral position, a pitch and a yaw of the central drive unit to thereby adjust a respective pitch and yaw of the surgical instruments and endoscope based on operator input, and to adjust a location of a remote center of motion along an axis of the insertion tube to maintain the remote center of motion at the incision location while allowing a length of the insertion tube that extends inside the patient to be adjusted to thereby adjust an insertion depth of the surgical instruments and endoscope in a surgical space within the patient based on operator input.

### BRIEF DESCRIPTION OF THE DRAWINGS

FIG. 1 is a perspective view of a robotic surgical system in an example deployed configuration.

FIG. 2 is a perspective view of the robotic surgical system of FIG. 1 in a stowed configuration.

FIG. 3 is a front view of the robotic surgical system of FIG. 1 in the stowed configuration.

FIG. 4 is a side view of the robotic surgical system of FIG. 1 in the stowed configuration.

FIG. 5 is a side view of the robotic surgical system of FIG. 1 in an example deployed configuration.

FIG. 6 is a front view of the robotic surgical system of FIG. 1 in an example deployed configuration.

FIG. 7 is a side view of the robotic surgical system of FIG. 1 in an example deployed configuration.

FIG. 8 is a top view of the robotic surgical system of FIG. 1 in an example deployed configuration.

FIG. 9 is a top view of the robotic surgical system of FIG. 1 in an example deployed configuration.

FIG. 10 is a top view of the robotic surgical system of FIG. 1 in an example deployed configuration.

FIG. 11 is a perspective partial view of the robotic surgical system of FIG. 1 in an example deployed configuration.

FIGS. 12A-12B are partial cross-sectional views of a portion of the robotic surgical system of FIG. 1.

FIG. 13 shows perspective views of variations of the robotic surgical system of FIG. 1.

FIG. 14 shows side views of the variations in FIG. 13 of the robotic surgical system of FIG. 1.

FIG. 15 is a perspective view of a robotic surgical system in an example deployed configuration.

FIG. 16 is a perspective view of the robotic surgical system of FIG. 15 in a stowed configuration.

FIG. 17 is a front view of the robotic surgical system of FIG. 15 in the stowed configuration.

FIG. 18 is a side view of the robotic surgical system of FIG. 15 in the stowed configuration.

FIG. 19 is a side view of the robotic surgical system of FIG. 15 in an example deployed configuration.

FIG. 20 is a front view of the robotic surgical system of FIG. 15 in an example deployed configuration.

FIG. 21 is a side view of a portion of the robotic surgical system of FIG. 15 depicting the adjustability of the insertion tube depth.

FIG. 22 is a top view of the robotic surgical system of FIG. 15 in an example deployed configuration.

FIG. 23 is a perspective partial view of the robotic surgical system of FIG. 15 in an example deployed configuration.

FIG. 24 shows a side view of a variations of the robotic surgical system of FIG. 15.

### DETAILED DESCRIPTION

SCARA Robotic Surgical System

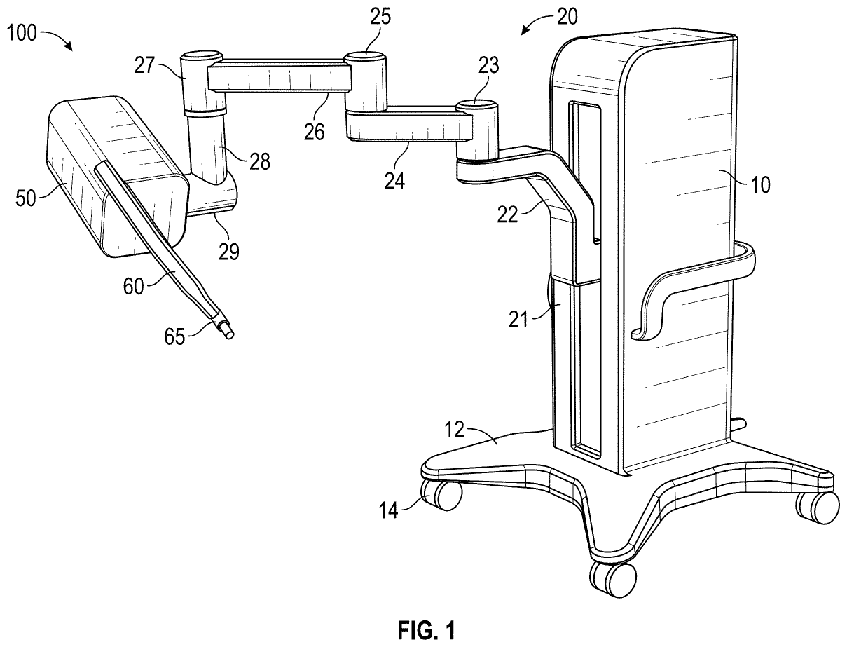

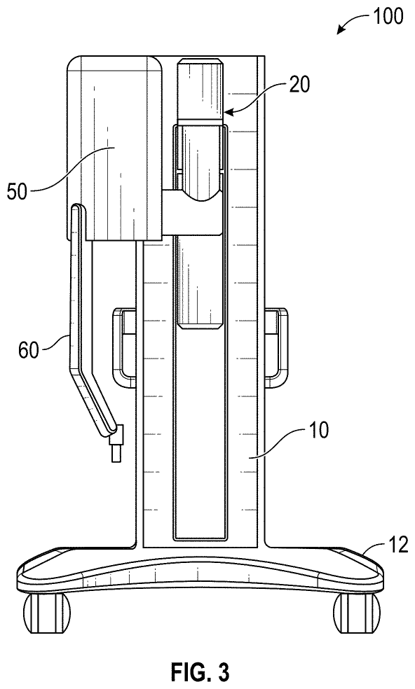

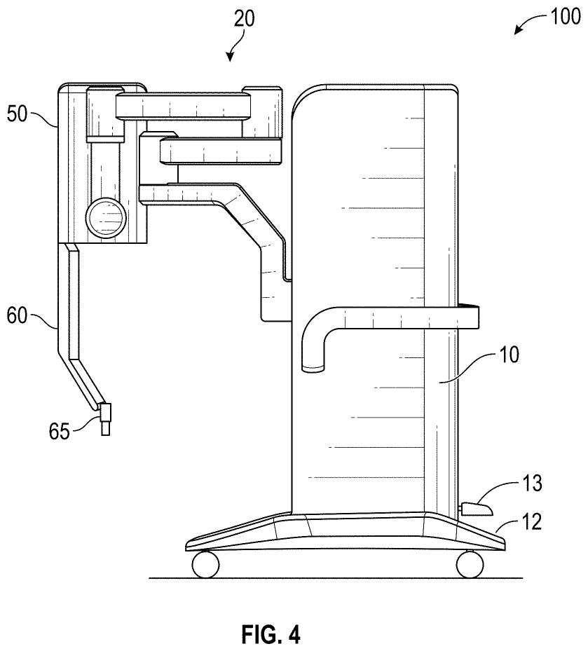

FIGS. 1-12B show a robotic surgical system 100. The system 100 includes a cart 10 (e.g., patient cart) with a base 12. The base 12 has one or more wheels 14 that allow the cart 10 (e.g., tower) to move along a surface (e.g., floor of the operating room). The wheels 14 can be selectively locked and unlocked (e.g., via a foot pedal 13, shown in FIG. 4). The system also includes an arm assembly 20 that is movably coupled to the cart 10, as further described below. The arm assembly 20 is a robotic (e.g., electromechanically controlled) arm assembly. A central drive unit (CDU) 50 is movably coupled to the arm assembly 20. One or more robotic surgical tools (not shown), such as surgical instruments and an endoscope, can be removably coupled to the central drive unit 50, which can extend at least a portion of the robotic surgical tools through a cannula or insertion tube 65 attached to a support arm 60 mechanically coupled (e.g., fixed to) the central drive unit 50 into a surgical space in a patient and operate end effectors of the robotic surgical tools to perform a surgical procedure within the surgical space.

In the illustrated implementation, the arm assembly 20 is a selective compliance assembly robot arm (SCARA) assembly. The arm assembly 20 has a first arm 22 movably attached to the cart 10 (e.g., to a side of the cart 10). In the illustrated embodiment, a proximal portion 21 of the first arm 22 can move (e.g., vertically) relative to the base 12 to vary a vertical position of the first arm 22 and thereby the arm assembly 20. An opposite end of the first arm 22 is movably (e.g., rotatably, pivotally) coupled to a second arm 24 via a joint 23 (e.g., first rotatable joint). An opposite end of the second arm 24 is movably (e.g., rotatably, pivotally) coupled to a third arm 26 via a joint 25 (e.g., second rotatable joint). An opposite end of the third arm 26 is movably (e.g., rotatably, pivotally) coupled to a fourth arm 28 via a joint 27 (e.g., third rotatable joint or yaw joint). An opposite end of the fourth arm 28 can be movably (e.g., rotatably, pivotally) coupled to the central drive unit (CDU) 50 via a joint 29 (e.g., fourth rotatable joint or pitch joint).

With continued reference to FIGS. 1-12B, the cart 10 can include one or more electric motors, drive units, transmissions and/or encoders to effect a vertical motion of the proximal portion 21 (and thereby the first arm 22 and arm assembly 20) relative to the base 12. In one implementation, the base 12 can have 1:1 wheel aspect ratio that inhibits (e.g., prevents) tip-over of the cart 10 through the full range of motion of the arm assembly 20. Drive units for effecting vertical and/or horizontal motion can include a harmonic drive, a planetary gearbox, belt drives, cable and pulley drives and worm and wheel drives.

Advantageously, the arm assembly 20 is horizontally constrained. Apart from the vertical adjustment of the arm assembly 20 and the pitch joint (joint 29), the degrees of freedom are naturally balancing. For example, second and third arms 24, 26 extend linearly (e.g., horizontally or transverse to an axis of the cart 10) and move only horizontally (e.g., relative to each other and relative to the first arm 22). The first arm 22 has at least a portion that extends horizontally (e.g., relative to each other and relative to the first arm 22) and can move vertically via the proximal portion 21, as discussed above. Each of the joints 23, 25, 27 and 29 can be robotic (e.g., electromechanically controlled), as discussed further below. The joint 27 (e.g., third rotatable joint) effects a yaw movement of the central drive unit 50, and the joint 29 (e.g., fourth rotatable joint) effects a pitch movement of the central drive unit 50. The arm assembly 20 can be operated to control the horizontal (e.g., proximal-distal) translation of the central drive unit 50. In the illustrated embodiment, the surgical system 100 has five degrees of freedom provided by the vertical motion of the first arm 22 and the four joints 23, 25, 27, 29 (e.g., excluding any degrees of freedom in the robotic surgical tools that are coupled to the central drive unit 50).

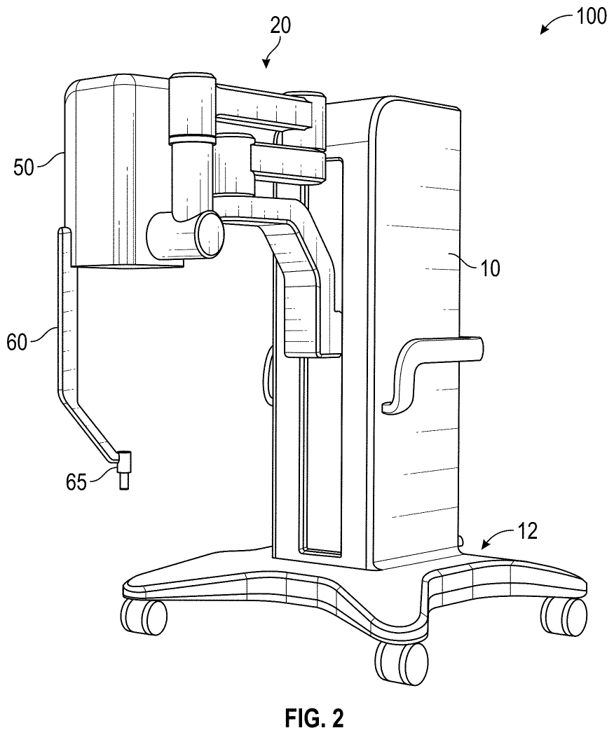

FIGS. 2-4 show the robotic surgical system 100 in a stowed configuration. Advantageously, the arm assembly 20 allows the robotic surgical system 100 to be moved into a compact assembly (e.g., for storage and/or movement between operating rooms). As best shown in FIG. 4, the first, second and third arms 22, 24, 26 can be arranged over each other (e.g., so that the arms 22, 24, 26 are aligned over each other), thereby achieving a compact arrangement. The second arm 24 has a shorter length than the third arm 26, allowing the second arm 24 to fit under the third arm 26 when in the compact position (e.g., where the third arm 26 is aligned with, or disposed over, the second arm 24 along the length of the second arm 24).

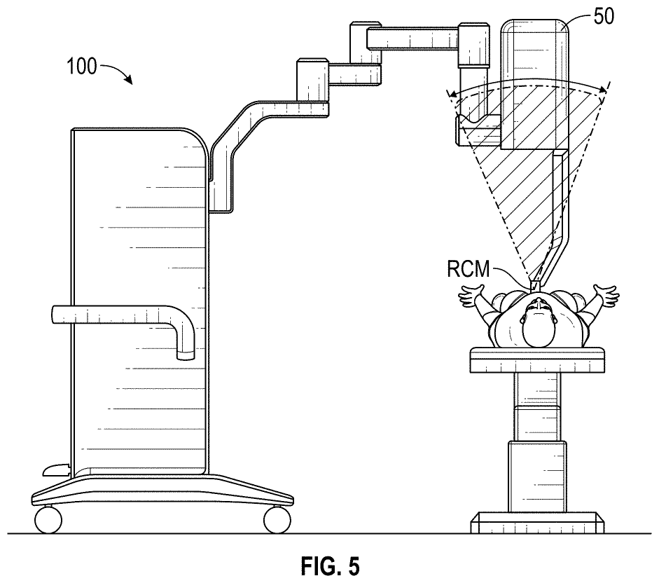

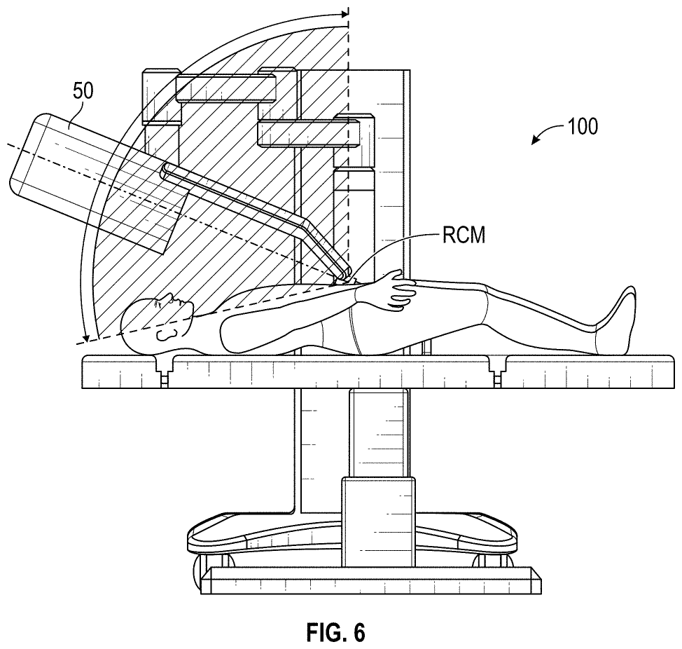

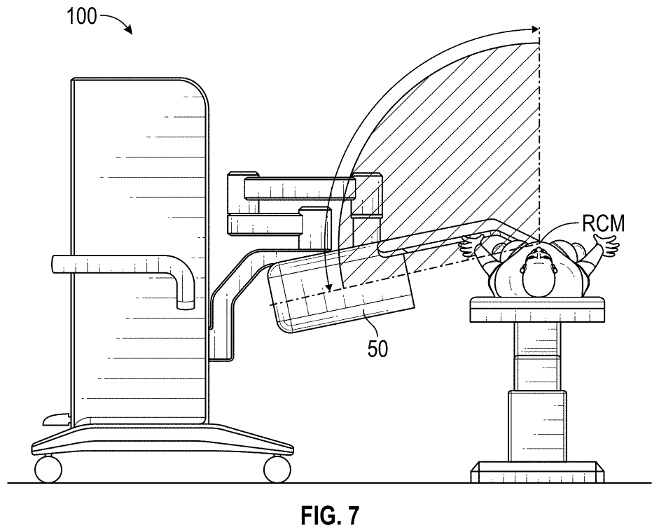

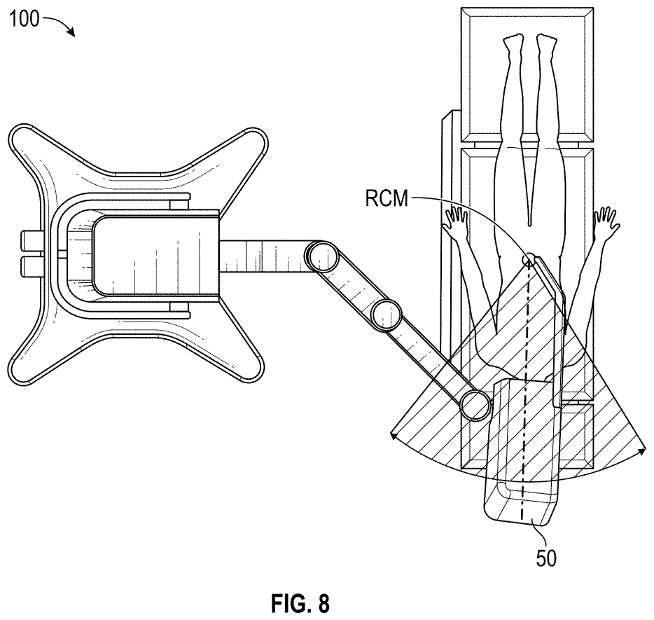

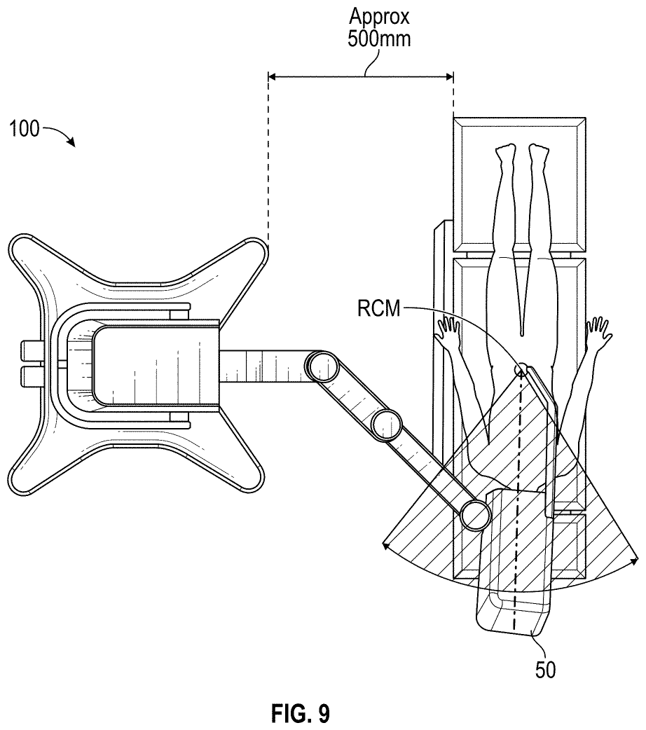

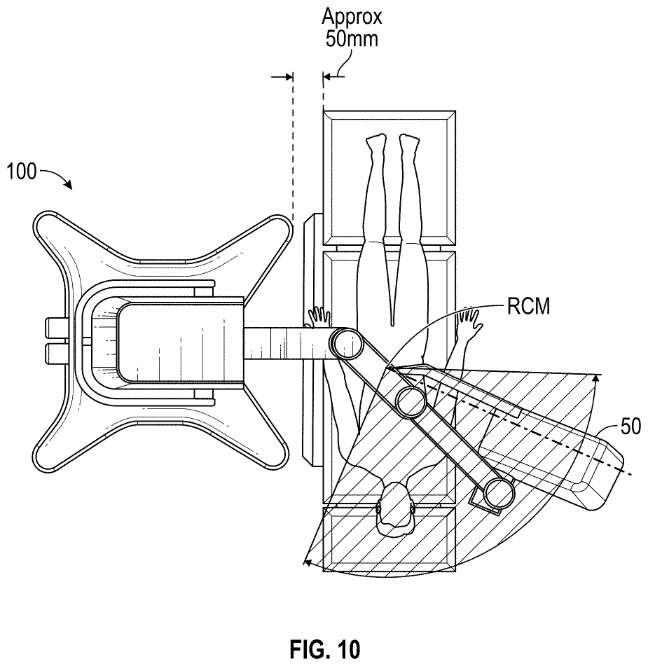

With reference to FIG. 5, the robotic surgical system 100 is shown in one orientation that provides for a range of motion from vertical of ±30° pitch and ±30° yaw (e.g., about the remote center of motion or RCM) in one implementation. In another implementation, the robotic surgical system 100 provides for a range of motion from vertical of ±90° pitch and ±180° yaw (e.g., about the remote center of motion or RCM). With reference to FIG. 6, the robotic surgical system 100 is shown in another orientation that provides for a range of motion from horizontal of between −10° to +90° pitch and of 120° of yaw (e.g., about the remote center of motion or RCM), as further described below. With reference to FIG. 7, the robotic surgical system 100 is shown in another orientation that provides for a range of motion from horizontal of between −10° to +90° pitch and of 120° of yaw (e.g., about the remote center of motion or RCM), as further described below. With reference to FIG. 8, the robotic surgical system 100 is shown in another orientation that provides for a range of motion from horizontal of between −10° to +90° pitch and of 60° of yaw (e.g., about the remote center of motion or RCM), as further described below. With reference to FIG. 9, the robotic surgical system 100 is shown in another orientation that provides for a range of motion of ±30° yaw (or total of 60° of yaw) (e.g., about the remote center of motion or RCM), where the base 12 of the cart 10 is approximately 500 mm from the operating table. With reference to FIG. 10, the robotic surgical system 100 is shown in another orientation that provides for a range of motion of between −60° and +90° yaw (or total of 120° of yaw), such as relative to an axis of the operating table (e.g., about the remote center of motion or RCM), where the base 12 of the cart 10 is approximately 50 mm from the operating table.

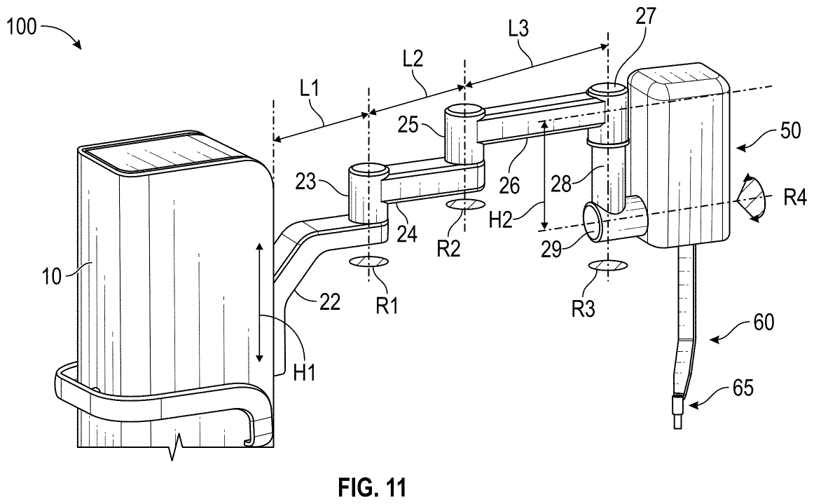

FIG. 11 shows a portion of the robotic surgical system 100 with the arm assembly 20 fully extended. The arm assembly 20 can move vertically relative to the cart 10 along a distance H1. In one implementation, the distance H1 can be between 800 mm and 900 mm, such as about 850 mm, and can allow for pitch rotation of between −10° to +90° relative to vertical. The first arm 22 can have a horizontal length L1, the second arm 24 can have a horizontal length L2, the third arm 26 can have a horizontal length L3, and the fourth arm 28 can have a length (e.g., vertical length or height) H2. The length L1 can be between 400 mm and 500 mm, such as about 480 mm. The length L2 can be between 300 mm and 400 mm, such as about 370 mm. The length L3 can be between 500 mm and 600 mm, such as about 560 mm. The length H2 can be between 300 mm and 400 mm, such as about 380 mm. The joint 23 (e.g., first rotatable joint) can in one implementation have a range of rotation R1 that is 360° (i.e., can fully rotate about the axis of the joint 23). The joint 25 (e.g., second rotatable joint) can in one implementation have a range of rotation R2 that is 360° (i.e., can fully rotate about the axis of the joint 25). The joint 27 (e.g., third rotatable joint) can in one implementation have a range of rotation R3 that is approximately 300° (yaw motion over a range of about 300°, such as with an internal hard stop in the joint 27). The joint 29 (e.g., fourth rotatable joint) can in one implementation have a range of rotation R4, or pitch motion over a range, of between-10° and about +90° (e.g., relative to horizontal, such as with an internal hard stop in the joint 29).

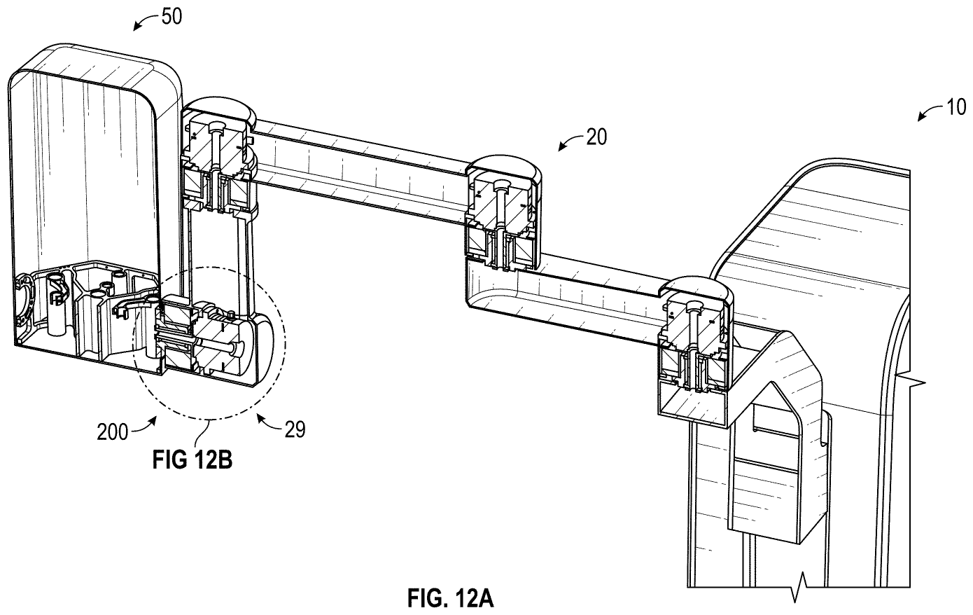

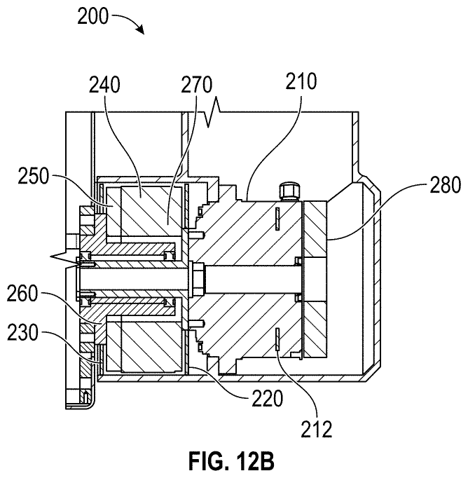

FIGS. 12A-12B show an example robotic drive system 200. Though FIG. 12A shows the drive system 200 in the joint 29 (e.g., fourth rotatable joint, or pitch joint), one of skill in the art will recognize that the drive system can be used in one or more (e.g., all) of the joints 23, 25, 27, 29 to control proximal-distal translation, pitch and/or yaw of the central drive unit 50. The robotic drive system 200 can include an electric motor assembly 210. In one implementation, the electric motor assembly 210 can include a brushless direct current (DC) motor for high efficiency and long service life and a harmonic drive for compactness, high torque capability, repeatability and minimal or no backlash. The system 200 can also include an encoder 212 to a known motor position, an encoder 220 to a known drive output position, and an encoder 230 to a known central drive unit 50 position when driven manually. The robotic drive system 200 can also include a brake (or clutch) 240 with a brake surface 250 that is engaged during drive motion or when in a static position or powered off (e.g., due to permanent magnets), which can then be released for manual operation. The brake 240 has a side 260 that is attached to a hub of the central drive unit 50 and is free to rotate whenever the drive system 200 is powered. The brake 240 also has a side 270 attached to the drive output. The robotic surgical system 200 also includes a safety brake 280 that engages when an emergency stop is actuated or when the robotic drive system 200 is powered off. The robotic surgical system 200 can operate in manual mode and robotic mode. In manual mode, the geartrain and motor have to be decoupled from the joint rotation, for example with a clutch. However, this can increase the mass of the joint.

In another implementation, the robotic surgical system 200 in one or more (e.g., all) of the joints 23, 25, 27, 29 can have a torque sensor T instead of the brake (or clutch) 240 so that the robotic surgical system 200 is a force-follow system to accommodate a robotic mode of operation and manual mode of operation without having to decouple the drive system during manual mode; rather, the drive system works with the operator (e.g., pushing on one or more of the first, second, third and fourth arms 22, 24, 26, 28 or one or more joints 23, 25, 27, 29). Additionally, the force-follow implementation advantageously allows active counterbalancing of the pitch joint (e.g., joint 29) at any configuration of the central drive unit 50. The robotic surgical system 200 with force-follow implementation can advantageously be approximately 40% lighter (e.g., have 40% of the mass) relative to the robotic surgical system 200 without force-follow (e.g., including a brake or clutch).

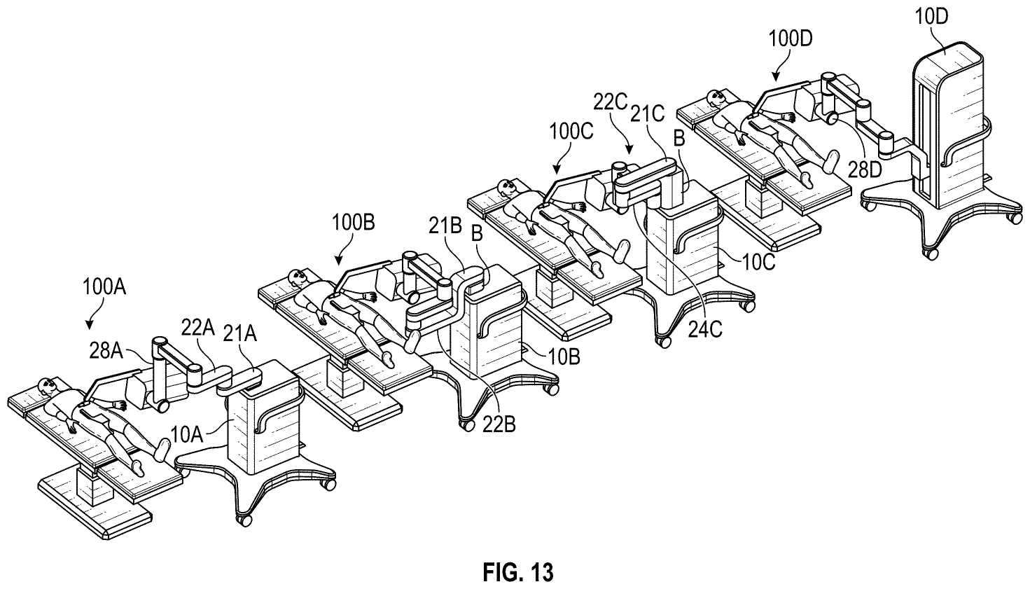

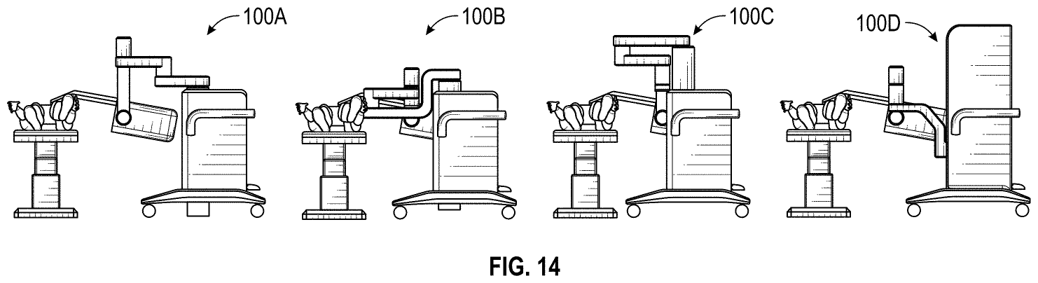

FIGS. 13-14 show example robotic surgical systems 100A-100D that are variations of the robotic surgical system 100. The features of the robotic surgical systems 100A-100D are similar to features of the robotic surgical system 100 in FIGS. 1-12B. Thus, reference numerals used to designate the various components of the robotic surgical systems 100A-100D are identical to those used for identifying the corresponding components of the robotic surgical system 100 in FIGS. 1-12B, except that an “A”, “B”, “C”, or “D” has been added to the numerical identifier. Therefore, the structure and description for the various features of the robotic surgical system 100 in FIGS. 1-12B are understood to also apply to the corresponding features of the robotic surgical systems 100A-100D in FIGS. 13-14, except as described below.

The robotic surgical system 100A differs from the robotic surgical system 100 in that the proximal portion 21A of the first arm 22A is attached to a boom arm (not shown) that extends (e.g., telescopically) from the top of the cart 10A, and the fourth arm 28A is longer than the fourth arm 28. Also, the cart 10A is smaller (e.g., shorter) than the cart 10.

The robotic surgical system 100B differs from the robotic surgical system 100 in that the proximal portion 21B of the first arm 22B is attached to a boom arm B that extends (e.g., telescopically) from the top of the cart 10B, and first arm 22B can be a “dogleg” arm (e.g., having two horizontal sections spaced vertically apart from each other). Also, the cart 10B is smaller (e.g., shorter) than the cart 10.

The robotic surgical system 100C differs from the robotic surgical system 100 in that the proximal portion 21C of the first arm 22C is attached to a boom arm B that extends (e.g., telescopically, not telescopically) from the top of the cart 10C, and the second arm 24C extends below the first arm 22C (e.g., inverse SCARA arrangement). Also, the cart 10C is smaller (e.g., shorter) than the cart 10.

The robotic surgical system 100D differs from the robotic surgical system 100 in that the cart 10D is taller than the cart 10, and the fourth arm 28D is shorter than the fourth arm 28 and be able to reach the operating table. In one example, the cart 10D can have the same height as the cart 10, but has a taller stowed position (e.g., than shown in FIGS. 2-4) because the cart 10D houses all the components that effect up/down motion of the arm assembly 20D statically. The fourth arm 28A-28D of the robotic surgical systems 100A-100D have different lengths to compensate for the different height of their respective cart 10A-10D and range of travel of the arm assembly 20A-20D relative to the cart 10A-10D, and still achieve the nominal remote center of motion (RCM) at the same height.

### Prismatic Robotic Surgical System

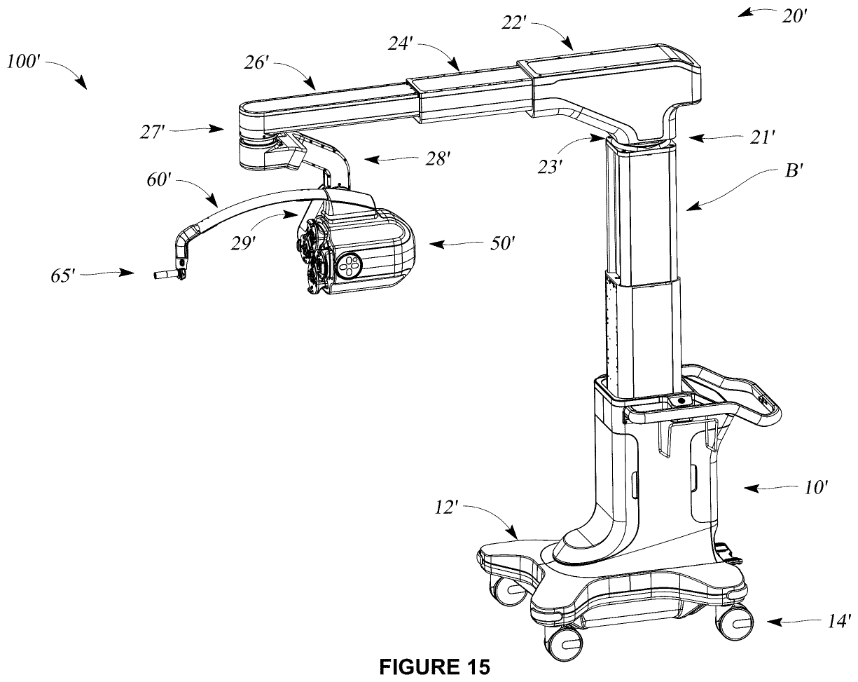

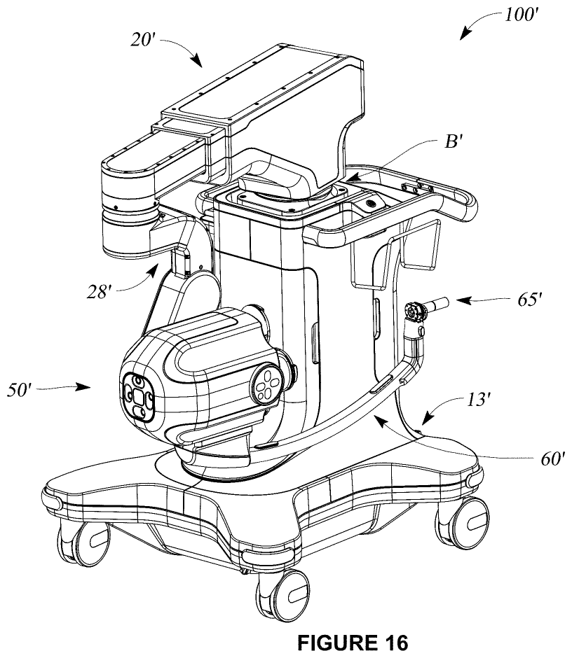

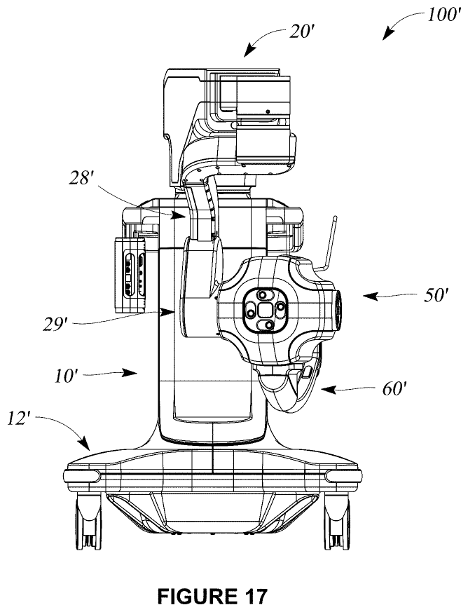

FIGS. 15-23 show a robotic surgical system 100′. The robotic surgical system 100′ includes a cart 10′ (e.g., patient cart) with a base 12′. The base 12′ has one or more wheels 14′ that allow the cart 10′ (e.g., tower) to move along a surface (e.g., floor of the operating room). The wheels 14′ can be selectively locked and unlocked (e.g., via a foot pedal 13′, shown in FIG. 16). The system also includes an arm assembly 20′ that is movably coupled to the cart 10′, as further described below. The arm assembly 20′ is a robotic (e.g., electromechanically controlled) arm assembly. A central drive unit (CDU) 50′ is movably coupled to the arm assembly 20′. One or more robotic surgical tools (not shown) can be removably coupled to the central drive unit 50′, which can extend at least a portion of the robotic surgical tools through a cannula or insertion tube 65′ attached to a support arm 60′ mechanically coupled (e.g., fixed to) the central drive unit 50′ into a surgical space in a patient and operate end effectors of the robotic surgical tools to perform a surgical procedure within the surgical space.

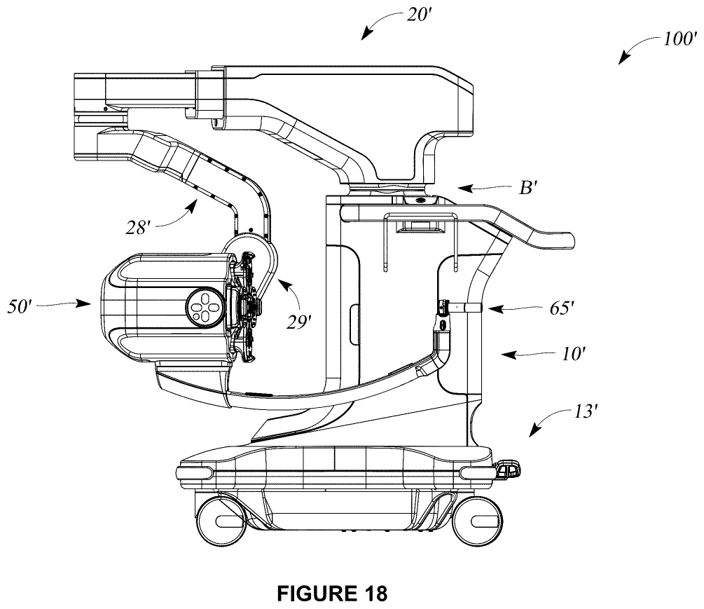

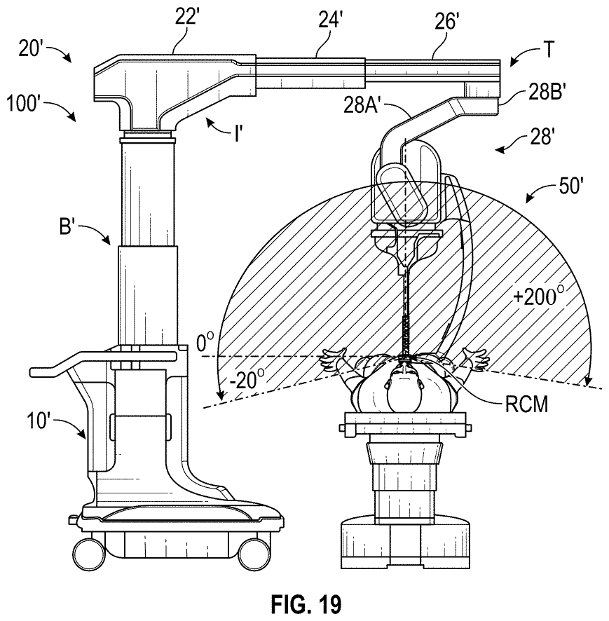

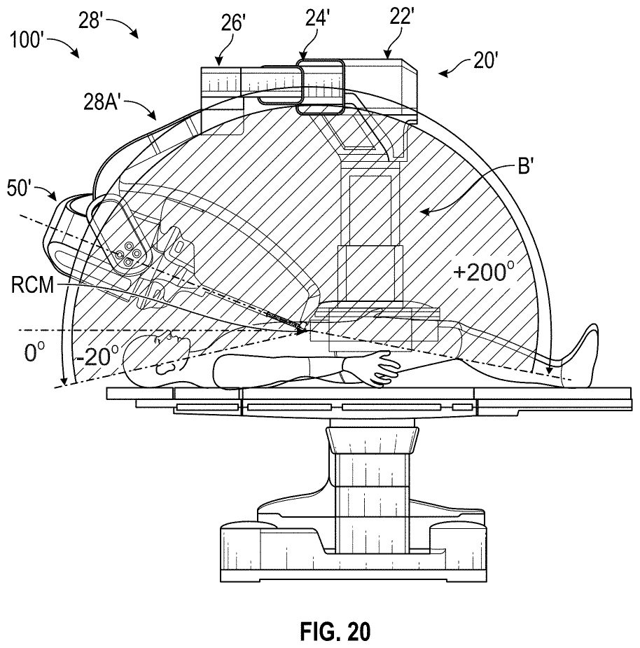

In the illustrated implementation, the arm assembly 20′ is a prismatic boom arm (e.g., a multi-stage prismatic boom arm) assembly. The arm assembly 20′ has a first arm section 22′ movably (e.g., rotatably, pivotally) coupled to a boom arm (e.g., pillar) B′ via a joint 23′ (e.g., first rotatable joint). The boom arm B′ is movably attached to the cart 10′ (e.g., to a top side of the cart 10′) so that the boom arm B′ can move relative to the cart 10′ to adjust a height of the arm assembly 20′ relative to the base 12′ to vary a vertical position (e.g., in the Z direction) of the arm assembly 20′. The arm assembly 20′ can include a second arm section 24′ movably coupled to the first arm section 22′ in a telescoping manner (e.g., at least a portion of the second arm section 24′ extends from within the first arm section 22′). The arm assembly 20′ can include a third arm section 26′ movably coupled to the second arm section 24′ in a telescoping manner (e.g., at least a portion of the third arm section 26′ extends from within the second arm section 24′). A fourth arm section 28′ can be movably coupled to the third arm section 26′ at an opposite end thereof via a joint 27′ (second rotatable joint or yaw joint) from the second arm section 26′. The central drive unit 50′ can be movably (e.g., rotatably, pivotally) coupled to the fourth arm section 28′ via a joint 29′ (e.g., third rotatable joint or pitch joint). The first, second and third arm sections 22′, 24′, 26′ can generally extend along the same axis, and can extend generally perpendicular to an axis of the boom arm B′ (e.g., extend generally horizontal or parallel to a support surface on which the base 12′ sits). In another implementation, the arm assembly 20′ can have two telescoping arm sections (instead of three) between the proximal portion 21′ and the fourth arm section 28′. In still another implementation, the arm assembly 20′ can have more than three telescoping arm sections (e.g., four, five). Advantageously, the fourth arm section 28′ or support arm for the central drive unit 50′ is shaped to allow the robotic surgical system 100′ to stow in a compact position, as shown in FIG. 18. The shape of a support arm portion 28A′ and a second support arm portion 28B′ (see FIGS. 19-20) of the fourth arm section 28′ can have a similar profile to an inner facing portion I′ of the arm assembly 20′, and facilitate the compact stowing profile (see FIG. 18). Additionally, said similar shapes between the fourth arm section 28′ and the inner facing portion I′ of the arm assembly 20′ inhibits (e.g., prevents) collision between the fourth arm section 28′ or support arm for the central drive unit 50′ and the arm assembly 20′ during operation of the robotic surgical system 100′.

The arm assembly 20′ includes electric motors, brakes and/or encoders for rotational motion of the arm assembly 20′ (e.g., first arm section 22′) relative to the boom arm B′ and for proximal-distal translation of the first, second and third arm sections 22′, 24′, 26′. In one implementation, the arm assembly 20′ can include a linear drive. For example, the linear drive can be a 1.5 Nm stepper motor that powers a timing belt drive. In other implementations the linear drive can have other configurations. In one implementation, one or more (e.g., all) of the joints 23′, 27′, 29′ (e.g., first rotatable joint, second rotatable joint or yaw joint, and third rotatable joint or pitch joint) can include a robotic drive system, such as the robotic drive system 200 described above in connection with FIGS. 12A-12B, for maintaining or adjusting the pitch and yaw of the central drive unit 50′. Drive units for effecting pitch and/or yaw motion can include a harmonic drive, a planetary gearbox, belt drives, cable and pulley drives and worm and wheel drives. Drive units for effecting the horizontal (proximal-distal) motion of the arm assembly 20′ can include belt drives, rack and pinions, lead screws, cables and pulleys and linear actuators.

With continued reference to FIGS. 15-23, the cart 10′ can include one or more electric motors, drive units, transmissions brakes and/or encoders to effect a vertical motion of the boom arm B′ relative to the base 12′. In one implementation, the base 12′ can have 1:1 wheel aspect ratio that inhibits (e.g., prevents) tip-over of the cart 10 through the full range of motion of the arm assembly 20′.

Advantageously, the arm assembly 20′ is horizontally constrained. Apart from the vertical adjustment of the arm assembly 20′ and the pitch joint (joint 29′), the degrees of freedom are naturally balancing. For example, first, second and third arm sections 22′, 24′, 26′ extend linearly (e.g., horizontally or transverse to an axis of the cart 10′) and move only horizontally (e.g., relative to each other). The first, second and third arm sections 22′, 24′, and 26′ can move relative to each other between an extended configuration (shown in FIG. 15) and a collapsed or retracted or stowed configuration (shown, for example, in FIG. 18), as further described below. Each of the joints 23′, 27′, and 29′ can be robotic (e.g., electromechanically controlled), as discussed further below. The joint 27′ (e.g., second rotatable joint) effects a yaw movement of the central drive unit 50′, and the joint 29′ (e.g., third rotatable joint) effects a pitch movement of the central drive unit 50′. The arm assembly 20′ can be operated to control the horizontal (e.g., proximal-distal) translation of the central drive unit 50′. In the illustrated embodiment, the surgical system 100′ has five degrees of freedom provided by the vertical motion of the boom arm B′, the horizontal motion of the first, second and third arm sections 22′, 24′, 26′ and the three joints 23′, 27′, 29′ (e.g., excluding any degrees of freedom in the robotic surgical tools that are coupled to the central drive unit 50′).

FIGS. 16-18 show the robotic surgical system 100′ in a stowed configuration. Advantageously, the arm assembly 20′ allows the robotic surgical system 100′ to be moved into a compact assembly (e.g., for storage and/or movement between operating rooms). As best shown in FIG. 18, each of the second and third arm sections 24′, 26′ can be at least partially retracted (e.g., mostly retracted, fully retracted) within the first arm section 22′, thereby achieving a compact arrangement (e.g., horizontally compact arrangement). Further, the boom arm B′ can be lowered relative to the cart (or tower) 10′ (e.g., to its lowest vertical position), to reduce the vertical height (in the Z direction) of the arm assembly 20′ relative to the base 12′, thereby achieving a compact arrangement (e.g., vertically compact arrangement). At least a portion of the fourth arm section 28′ can have an L-shaped profile between the joint 27′ and joint 29′, allowing the central drive unit 50′ to be oriented vertically (as shown in FIGS. 16-18) and proximate a side surface of the arm assembly 20′ (e.g., side surface of the third arm section 26′).

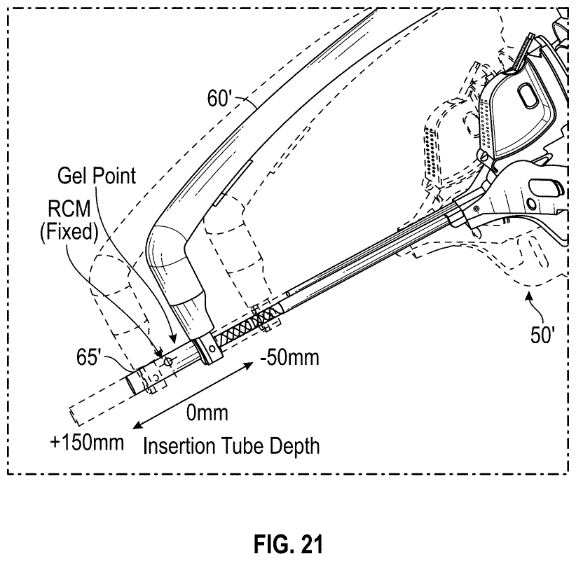

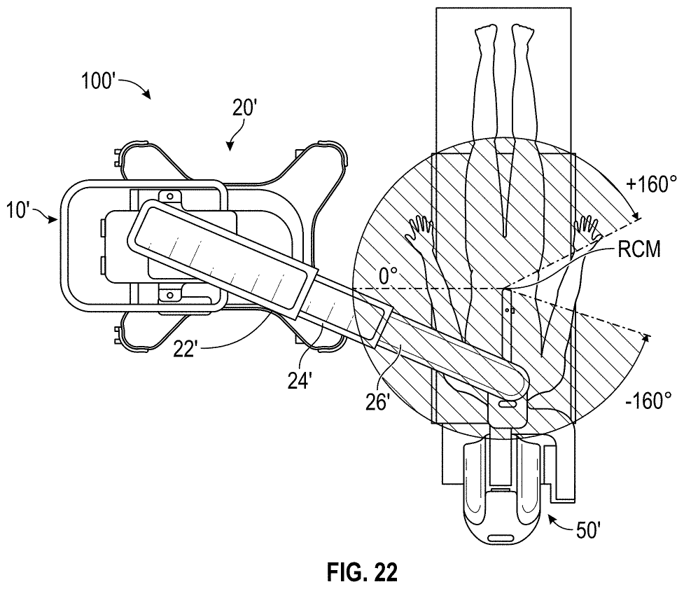

With reference to FIG. 19, the robotic surgical system 100′ is shown in one orientation that provides for a range of motion from horizontal of −20° to +200° pitch (e.g., about the remote center of motion or RCM). With reference to FIG. 20, the robotic surgical system 100′ is shown in another orientation that provides for a range of motion from horizontal of between −20° to +200° pitch (e.g., about the remote center of motion or RCM), as further described below. With reference to FIG. 21, the robotic surgical system 100′ is operable to adjust an insertion tube depth (e.g., an amount the insertion tube 65′ is inserted into the patient). In one example, the robotic surgical system 100′ is operable to adjust the insertion tube depth within a range of −50 mm and +150 mm relative to a nominal insertion depth position—that is between a position up to 50 mm retracted away from the remote center of motion (e.g., at the incision location) and a position up to 150 mm further inserted into the patient (e.g., and relative to the remote center of motion or incision point). In one implementation, the adjustment in the insertion depth of the insertion tube 65′ is provided via operation of the arm assembly 20′, such as adjustment in the length of the first arm section 22′, second arm section 24′ and/or third arm section 26′. With reference to FIG. 22, the robotic surgical system 100′ is shown in another orientation that provides for a range of motion from horizontal of between −160° to +160° of yaw (e.g., about the remote center of motion or RCM). That is, the robotic surgical system 100′ can have a range of motion of −20° to +200° of pitch and −160° to +160° of yaw. The robotic surgical system 100′ advantageously can be placed at varying distances from the operating table. In one implementation the base 12′ of the cart 10′ can be approximately 600 mm from the operating table. In another implementation, the base 12′ of the cart 10′ can be approximately 50 mm from the operating table.

In one implementation, the robotic surgical system 100′ can have a torque sensor T proximate the joint 27′ (e.g., disposed between the third arm section 26′ and the fourth arm section or support arm 28′) so that the robotic surgical system 100′ is a force-follow system to accommodate a robotic mode of operation and manual mode of operation without having to decouple the drive system during manual mode; rather, the drive system works with the operator (e.g., pushing on one or more of the first, second, third and fourth arms 22′, 24′, 26′, 28′ or actuating one or more of the joints 23′, 25′, 27′, 29′). Additionally, the force-follow implementation advantageously allows active counterbalancing of the pitch joint (e.g., joint 29′) at any configuration of the central drive unit 50′. The robotic surgical system 100′ with force-follow implementation can advantageously be approximately 40% lighter (e.g., have 40% of the mass) relative to the robotic surgical system 100′ without force-follow (e.g., including a brake or clutch).

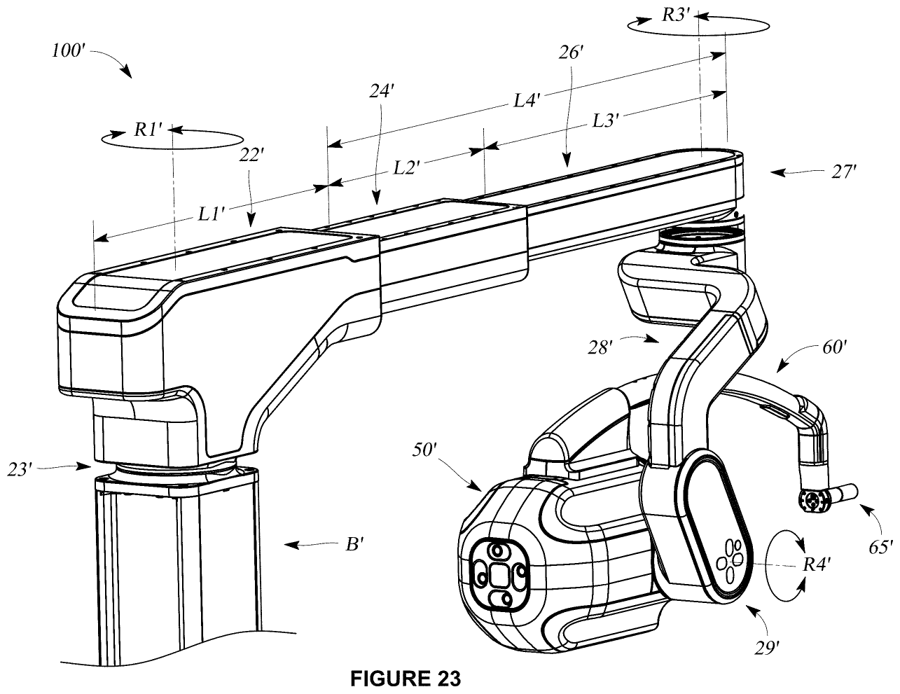

FIG. 23 shows a portion of the robotic surgical system 100′ with the arm assembly 20′ fully extended. The arm assembly 20′ can move vertically relative to the cart 10′ along a distance H1′ (e.g., via movement of the boom arm B′ relative to the cart 10′). In one implementation, the distance H1′ can be between 800 mm and 1200 mm, such as about 1100 mm. The first arm section 22′ can have a horizontal length L1′, the second arm section 24′ can have a horizontal length L2′, and the third arm section 26′ can have a horizontal length L3′. The length L1′ can be between 700 mm and 800 mm, such as about 750 mm. The length L2′ can be between 700 mm and 800 mm, such as about 750 mm. The length L3′ can be between 600 mm and 700 mm, such as about 650 mm. In one implementation, the horizontal travel distance L4′ of the second arm section 24′ and third arm section 26′ relative to the end of the first arm section 22′ is approximately between about 800 mm and about 1000 mm, such as about 840 mm. The joint 23′ (e.g., first rotatable joint) can in one implementation have a range of rotation R1′ that is 120° (e.g., +60° to −60° to horizontal, such as with an internal hard stop in the joint 23′). The joint 27′ (e.g., second rotatable joint) can in one implementation have a range of rotation R3′, or yaw motion over a range, of 320° (i.e., can rotate between-160° and +160° about the axis of the joint 27′). The joint 29′ (e.g., third rotatable joint) can in one implementation have a range of rotation R4′, or pitch motion over a range, of between +20° and about −200° (e.g., relative to horizontal, such as with an internal hard stop in the joint 29′).

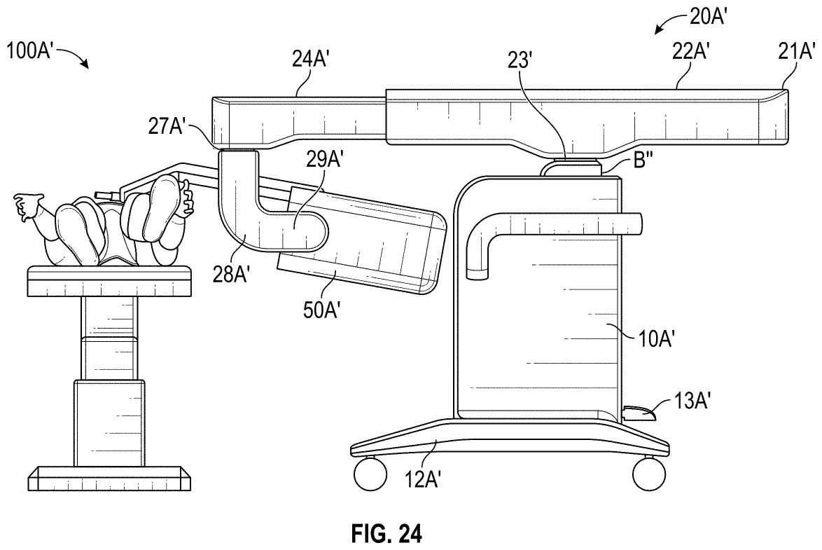

FIG. 24 shows an example robotic surgical system 100A′ that is a variation of the robotic surgical system 100′. The features of the robotic surgical system 100A′ are similar to features of the robotic surgical system 100′ in FIGS. 15-23. Thus, reference numerals used to designate the various components of the robotic surgical system 100A′ are identical to those used for identifying the corresponding components of the robotic surgical system 100′ in FIGS. 15-23, except that an “A” has been added to the numerical identifier. Therefore, the structure and description for the various features of the robotic surgical system 100′ in FIGS. 15-23 are understood to also apply to the corresponding features of the robotic surgical systems 100A′ in FIG. 24, except as described below.

The robotic surgical system 100A′ differs from the robotic surgical system 100′ in that the arm assembly 20A′ includes two stages—a first arm portion 22A′ and a second arm portion 24A′, with the arm portion 28A′ movably (e.g., rotatably, pivotally) coupled to the end of the second arm portion 24A′ via a joint 27A′. The central drive unit 50A′ movably (e.g., rotatably, pivotally) coupled to another end of the arm portion 28A′. Additionally, the joint 23A′ (first rotatable joint) is between the boom arm B″ and an intermediate portion of the first arm section 22A′ (i.e., not at the proximal portion 21A′ of the first arm section 22A′). Advantageously, the robotic surgical system 100′ has fewer mechanical drives and drive transmissions (e.g., due to there being one less arm portion or state in the prismatic arm assembly 20A′). Additionally, a counterbalance can optionally be incorporated with (e.g., attached to, housed within) the proximal portion 21A′ that overhangs relative to the joint 23A′.

### Method of Operation

With respect to the robotic surgical system 100, 100A-100D, 100′, 100A′, the remote center of motion (RCM) can be sufficient to reach sites in the human body to perform at least the following procedures: Hysterectomy, Cholecystectomy, Colectomy, Splenectomy, Partial Nephrectomy, Prostatectomy, Tongue base surgery. The remote center of motion (RCM) can be set at the beginning of the surgical procedure. The RCM can be a software implemented RCM, where the RCM can be moved along the axis of the insertion tube. For example, during the procedure the surgeon may prefer to have the surgical instruments deeper in the surgical space. To achieve this, the RCM can be modified via software, allowing the central drive unit 50, 50′, 50A′ to be moved closer to the patient (e.g., by the arm assembly 20, 20A-20D, 20′, 20A′) to move the insertion tube further into the surgical space but maintaining the pivot point of the insertion tube at the incision point.

The robotic surgical system 100, 100A-100D, 100′, 100A′ can operate in static, manual and robotic modes. In manual mode, the system 100, 100A-100D, 100′, 100A′ is active during setup and after the procedure. Under manual guidance, the arm assembly 20, 20A-20D, 20′, 20A′ is positioned by the operator to dock to the insertion tube. Gravity compensation is provided for non-horizontal degrees of freedom (e.g., vertical motion, pitch movement). The manual mode also allows the operator to return the system 100, 100A-100D, 100′, 100A′ into a compact stowed state.

In static mode, the arm assembly 20, 20A-20D, 20′, 20A′ is static when the surgeon is actively driving one or more of the surgical instruments and/or endoscope. All degrees of freedom of the arm assembly 20, 20A-20D, 20′, 20A′ are locked to provide a stable position for the surgical instruments and endoscope via the central drive unit 50, 50A-50D, 50′, 50A′. In a no power state (e.g., due to loss of power), the system 100, 100A-100D, 100′, 100A′ is static and in a locked state. The surgical instruments, endoscope and insertion tube can be manually retracted to allow the cart 10, 10A-10D, 10′, 10A′ to be moved away from the operating table.

In robotic mode, under surgeon control, the yaw and/or pitch angle of the central drive unit 50, 50A-50D, 50′, 50A′ can be adjusted robotically (e.g., to adjust the pitch and/or yaw of the surgical instruments and endoscope). The RCM remains fixed so there is no translation about the mid-abdominal wall. The RCM can be a software implemented RCM, where the RCM can be moved along the axis of the insertion tube, as described above.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Of course, the foregoing description is that of certain features, aspects and advantages of the present invention, to which various changes and modifications can be made without departing from the spirit and scope of the present invention. Moreover, the devices described herein need not feature all of the objects, advantages, features and aspects discussed above. Thus, for example, those of skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed devices.

What is claimed is:

1. A robotic surgical system, comprising:
a cart extending vertically above a base;
an arm assembly movably coupled to the cart, the arm assembly being selectively movable relative to the cart via a boom arm that connects the arm assembly to the cart to vary a height of the arm assembly relative to the base, the arm assembly pivotally coupled to the boom arm via a first joint and configured to pivot about a vertical axis through the first joint, the arm assembly comprising
a plurality of transverse arm sections extending perpendicular to the boom arm, each transverse arm section telescopically coupled to another of the transverse arm sections and operable by one or more actuators to linearly extend relative thereto between an extended position and a retracted position,
a support arm pivotally coupled to a last of the transverse arm sections about a second joint and extending downwardly therefrom; and
a central drive unit pivotally coupled to the support arm about a third joint, the central drive unit comprising one or more robotic surgical instruments and an endoscope removably coupled thereto, the surgical instruments and endoscope configured to extend through a single insertion tube configured to be inserted through an incision location in a patient,
wherein each of the joints and actuators is robotically controlled to adjust one or more of a lateral position, a pitch and a yaw of the central drive unit to thereby adjust a respective pitch and yaw of the surgical instruments and endoscope based on operator input, and to allow an insertion depth of the insertion tube when inserted in the patient to be adjustable to thereby adjust an insertion depth of the surgical instruments and endoscope in a surgical space within the patient based on operator input while maintaining a location of a remote center of motion at an incision location on the patient.

2. The robotic surgical system of claim 1, wherein the plurality of transverse arm sections include:
a first transverse arm section pivotally coupled to the boom arm and configured to rotate relative to the boom arm along a first plane,
a second transverse arm section telescopically coupled to the first transverse arm section and configured to move linearly relative to the first transverse arm section between a retracted position and an extended position relative to the first transverse arm section, and
a third transverse arm section telescopically coupled to the second transverse arm section and configured to move linearly relative to the second transverse arm section and the first transverse arm section between a retracted position and an extended position relative to the first and second transverse arm sections, the support arm being pivotally coupled to the third transverse arm section.

3. The robotic surgical system of claim 1, wherein each of the joints includes an electric motor operable to effect a pivoting motion of the joint about its axis to effect a motion to adjust one or more of the lateral position, the pitch and the yaw of the central drive unit to thereby adjust a respective pitch and yaw of the surgical instruments and endoscope based on operator input.

4. The robotic surgical system of claim 3, further comprising a brake or clutch operatively coupled to the electric motor and selectively operable to decouple the electric motor from the joint to allow for manual operation of the arm assembly and selectively operable to lock a position of the joint during operation of the surgical instruments or endoscope or when the robotic surgical system experiences loss of power.

5. The robotic surgical system of claim 3, further comprising a torque sensor, the electric motor configured to operate in a force-follow mode based on input from the torque sensor that senses an operator force on the joint.

6. The robotic surgical system of claim 5, wherein the torque sensor is disposed between the last of the transverse arm sections and the support arm.

7. The robotic surgical system of claim 2, wherein the arm assembly is configured to achieve a compact stowed configuration where the second transverse arm section is retracted relative to the first transverse arm section and the third transverse arm section is retracted relative to the second transverse arm section.

8. The robotic surgical system of claim 1, wherein the boom arm is a pillar that extends from a top end of the cart and is operable to vary a height of the arm assembly above the base by axially moving the pillar relative to the cart.

9. The robotic surgical system of claim 1, wherein the support arm has a shape corresponding to a shape of an inner facing portion of the arm assembly.

10. The robotic surgical system of claim 9, wherein the support arm has a support arm portion that extends at an angle corresponding to an angle of the inner facing portion of the arm assembly.

11. A robotic surgical system, comprising:
a cart extending vertically above a base;
an arm assembly movably coupled to the cart, the arm assembly being selectively movable relative to the cart to vary a height of the arm assembly relative to the base, the arm assembly constrained to move horizontally and comprising
a plurality of transverse arms extending perpendicular to the cart, each transverse arm pivotally coupled to another of the transverse arms via a joint and configured to pivot about a vertical axis through the joint,
a support arm pivotally coupled to a last of the transverse arms about a yaw joint and extending downwardly therefrom; and
a central drive unit pivotally coupled to the support arm about a pitch joint, the central drive unit comprising one or more robotic surgical instruments and an endoscope removably coupled thereto, the surgical instruments and endoscope configured to extend through a single insertion tube configured to be inserted through an incision location in a patient,
wherein each of the joint, yaw joint and pitch joint is robotically controlled to adjust one or more of a lateral position, a pitch and a yaw of the central drive unit to thereby adjust a respective pitch and yaw of the surgical instruments and endoscope based on operator input, and to allow an insertion depth of the insertion tube when inserted in the patient to be adjustable to thereby adjust an insertion depth of the surgical instruments and endoscope in a surgical space within the patient based on operator input while maintaining a location of a remote center of motion at an incision location on the patient.

12. The robotic surgical system of claim 11, wherein the plurality of transverse arms include:
a first transverse arm vertically movable relative to the cart and at least partially extending along a first plane,
a second transverse arm pivotally coupled to the first transverse arm about a first joint and extending along a second plane parallel to the first plane, the second transverse arm configured to pivot about a vertical axis through the first joint, and
a third transverse arm pivotally coupled to the second transverse arm about a second joint and extending along a third plane parallel to the first and second planes, the third transverse arm configured to pivot about a vertical axis through the second joint, the support arm pivotally coupled to the third transverse arm.

13. The robotic surgical system of claim 11, wherein each of the joints includes an electric motor operable to effect a pivoting motion of the joint about its axis to effect a motion to adjust one or more of the lateral position, the pitch and the yaw of the central drive unit to thereby adjust a respective pitch and yaw of the surgical instruments and endoscope based on operator input.

14. The robotic surgical system of claim 13, further comprising a brake or clutch operatively coupled to the electric motor and selectively operable to decouple the electric motor from the joint to allow for manual operation of the arm assembly and selectively operable to lock a position of the joint during operation of the surgical instruments or endoscope or when the robotic surgical system experiences loss of power.

15. The robotic surgical system of claim 13, further comprising a torque sensor, the electric motor configured to operate in a force-follow mode based on input from the torque sensor that senses an operator force on the joint.

16. The robotic surgical system of claim 11, wherein the arm assembly is configured to achieve a compact stowed configuration where one of the transverse arms overlaps with another of the transverse arms.

17. The robotic surgical system of claim 11, wherein the arm assembly is movably coupled to a side of the cart.

18. The robotic surgical system of claim 11, wherein the arm assembly is movably coupled to a top of the cart.
